(19) United States
(12) Patent Application Publication
(10) Pub. No.: US 2020/0005917 A1
(43) Pub. Date: Jan. 2, 2020

(54) SYSTEMS AND PROCESSES FOR TRIAGED DATA DISPLAY

(71) Applicants: John P. FORD, Unadilla, NY (US); Gustavo P. SUDRE, Washington, DC (US)

(72) Inventors: John P. FORD, Unadilla, NY (US); Gustavo P. SUDRE, Washington, DC (US)

(21) Appl. No.: 16/455,187

(22) Filed: Jun. 27, 2019

Related U.S. Application Data
(60) Provisional application No. 62/691,375, filed on Jun. 28, 2018.

(51) Int. Cl. G16H 15/00; G16H 10/60; G16H 80/00; A61B 5/00

(52) U.S. Cl. CPC G16H 15/00; G16H 10/60; A61B 5/7435; A61B 5/7445; G16H 80/00

(57) ABSTRACT

A triage process includes a computer directing a user triaged data display to appear on a user display monitor of a designated user. The process also includes the computer prompting the designated user through the user triaged data display to initiate a triage task associated with a report appearing on the user triaged data display. The process further includes the computer prompting the designated user through the user triaged data display to assign the triage task to a triage partner. The process additionally includes the computer directing an alert message associated with the triage task to appear as part of a partner triaged data display on a partner display monitor of the triage partner based on inputs from the designated user. A triaged data display system includes a display monitor and a computer operatively connected to the display monitor. The computer is configured to implement a triage process.

### CROSS-REFERENCE TO RELATED APPLICATIONS

This application claims priority to and the benefit of U.S. Provisional Application No. 62/691,375, filed Jun. 28, 2018, entitled “SYSTEMS AND PROCESSES FOR TRIAGED DATA DISPLAY”, which is hereby incorporated herein by reference in its entirety.

### FIELD OF THE INVENTION

The present embodiments are directed to the fields of tracking, monitoring, presentation, and/or display of information. More particularly, the present embodiments pertain to computer-implemented systems and methods that track and/or monitor activities and/or display their current status based on prioritized data from any size data universe.

### BACKGROUND OF THE INVENTION

In medicine, potentially-avoidable patient injuries and deaths unfortunately occur occasionally. These injuries and deaths may occur, in part, because critical test results are not recognized or acted upon quickly enough.

Critical results, in contrast to abnormal but not critical results, may get lost in an overwhelming volume of not critical results. Critical results may also be delivered directly to the patient who may not respond to them well or appropriately. Because these results may come from laboratories that are near the patient but that may not be linked electronically to the practitioner or primary health care provider, the practitioner may not even be aware of the results at all or until significantly later unless contacted by the patient.

Conventional medical software systems are often complex. Finding where critical information resides in such systems can be difficult. In conventional medical data systems, there is no simple way to establish that one problem or issue has been appropriately taken care of by a health care professional, whereas another problem or issue to be addressed by the same or another health care professional is still pending a resolution.

### BRIEF DESCRIPTION OF THE INVENTION

In exemplary embodiments, systems and processes enable generation and tracking of triage tasks and facilitate completion of these triage tasks. The designated user who has generated the triage task is able to easily follow and monitor the status of the triage task in real time in an ongoing basis through the triaged data display system and process.

In an embodiment, a triage process includes a computer directing a user triaged data display to appear on a user display monitor of a designated user. The process also includes the computer prompting the designated user through the user triaged data display to initiate a triage task associated with a report appearing on the user triaged data display. The process further includes the computer prompting the designated user through the user triaged data display to assign the triage task to a triage partner. The process additionally includes the computer directing an alert message associated with the triage task to appear as part of a partner triaged data display on a partner display monitor of the triage partner based on inputs from the designated user.

In another embodiment, a triaged data display system includes a user display monitor and a computer operatively connected to the user display monitor. The computer is configured to direct a user triaged data display to appear on the user display monitor of a designated user, prompt the designated user through the user triaged data display to initiate a triage task associated with a report appearing on the user triaged data display, prompt the designated user through the user triaged data display to assign the triage task to a triage partner, and direct an alert message associated with the triage task to appear as part of a partner triaged data display on a partner display monitor of the triage partner based on inputs from the designated user.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 shows a triage process in a triaged data display system of the present disclosure.

FIG. 2 shows drop-down menus for triage actions in an embodiment of the present disclosure.

FIG. 3 shows a triaged data display system in an embodiment of the present disclosure.

FIG. 4 shows a triaged data display in an embodiment of the present disclosure.

FIG. 5 shows a display format for data in an embodiment of the present disclosure.

FIG. 6 shows another display format for data in an embodiment of the present disclosure.

FIG. 7 schematically shows a side view of a virtual stack of patient data in an embodiment of the present disclosure.

FIG. 8 schematically shows the workflow in a triaged data display in medical data management from a top view of patient data stacks in an embodiment of the present disclosure.

FIG. 9 shows a user customization option of critical settings, critical categories, and critical magnitude of value changes for data in an embodiment of the present disclosure.

Wherever possible, the same reference numbers will be used throughout the drawings to represent the same parts.

### DETAILED DESCRIPTION OF THE INVENTION

Provided are systems and methods for establishing, tracking, and monitoring triaged data and presenting the data to a designated user in a manner that allows the designated user to easily determine the current status of the tracked and monitored triaged data.

Embodiments of the present disclosure, for example, in comparison to concepts failing to include one or more of the features disclosed herein, provide a display that represents all data on a single screen, provide a display that represents a triaged critical report on a screen until the triaged critical report is addressed, provide an invariant display layout, provide a system that interfaces with any software, provide a display that conforms to the human visual field, provide a system that works with any size database, provide a system that is adaptable for user-defined triage needs, provide a display with a simple format, provide a system with user-adjustable critical thresholds for triage on the fly, provide a system for generating a triaged task, provide a system for tracking the status of a triaged task, provide a system for completing a triaged task, or combinations thereof.

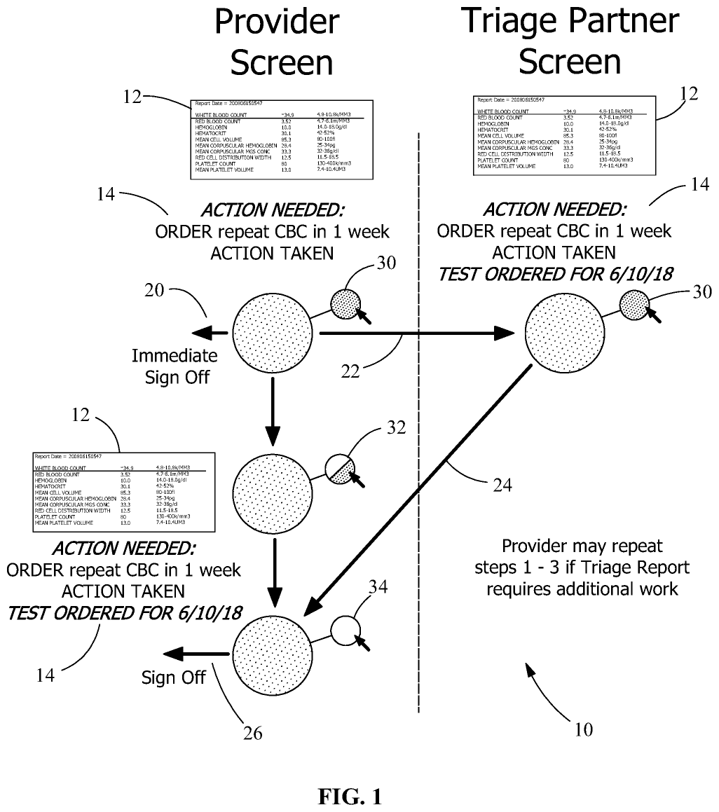

In exemplary embodiments, a triaged data display (TDD) includes a triage function, as shown schematically in FIG. 1 for a medical data application.

FIG. 1 exemplifies a triage process 10 enabling task triage within a triaged data display system. When a critical report 12 is issued for review by a designated user, the designated user has several options upon reviewing the critical report 12. A computer preferably prompts the designated user of the options throughout the triage process 10 through the triaged data display. Any appropriate prompting format on the triaged data display may be used by the computer, including, but not limited to, icons, buttons, drop-down menus, or cursor prompts. If the designated user feels that no further action is warranted, the designated user may immediately sign off 20 on the critical report 12. If the designated user feels that further action is needed, the designated user may input an alert message 14 into the triaged data display system, thereby generating a triage task. After the designated user has provided the alert message 14, the designated user may take the further action indicated in the alert message 14 and update the alert message 14 with information about the action having been taken.

Alternatively, the designated user may assign 22 the triage task to a triage partner, in which case the triage partner may view the critical report 12 and the alert message 14 on the triage partner's display screen. In some embodiments, the triage partner is another health care provider. In some embodiments, the triage partner is a health care specialist. When the triage partner takes the further action indicated in the alert message 14 and updates the alert message 14 with information about the action having been taken the triaged data display system updates 24 the designated user through the triaged data display that the action has been taken. Alternatively, the system may automatically update the alert message 14 upon completion of the triage task by the triage partner. The designated user may then sign off 26 on the critical report 12.

In exemplary embodiments, the triage process 10 is fully compliant with the Health Insurance Portability and Accountability Act (HIPAA) such that patient health data is only shared with authorization by the patient.

In some embodiments, the patient may act as the designated user or a triage partner. This arrangement would have several additional benefits. Because the patient is initiating the distribution of his or her clinical data, there would be no concerns about HIPAA compliance (and thus data confidentiality). In some embodiments where the patient is the designated user or a triage partner, the computer prompts the patient by way of the triaged data display to approve sharing of a critical report with a health care professional, who may not have received the critical report, such as, for example, the patient's primary care physician not receiving a critical report from a health care specialist for HIPAA-related reasons. Also there is a consistent data format for clinical result delivery over the internet that makes constructing an interface easy. In exemplary embodiments, all reports share a common display format. Finally, the practitioner or primary health care provider (or the designated triage partner) would have access to all patient results regardless of Electronic Health Record (EHR) compatibility across different EHR platforms.

In the process of FIG. 1, the shown critical report 12 is a complete blood count (CBC) medical report showing test results falling outside of a predetermined normal range. The triage task, as indicated by the alert message 14 in FIG. 1, is to repeat the CBC test, but any appropriate triage task may be entered by the designated user.

Upon generation of the triage task as a result of an input of an alert message 14 by the designated user, the triaged data display system displays a triage icon 30, in the form of a filled circle in FIG. 1, indicating that there is an outstanding triage task. When the designated user transfers the triage task to the triage partner, the triaged data display system displays the triage icon 30 on the triage partner's display screen and displays an in-progress icon 32, in the form of a half-filled circle in FIG. 1, on the designated user's display screen, indicating that the triage task has been assigned but not completed. At any time point during the pendency of the triage task, the designated user may easily find the status of the triage task by viewing the in-progress icon 32 on the display screen.

When the designated user or the triage partner completes the triage task, the triaged data display system displays a completed icon 34, in the form of an open circle in FIG. 1, indicating no outstanding triage tasks. The designated user may initiate a new triage task and the triage steps may be repeated, if the triage action results in another critical report or another unsatisfactory result.

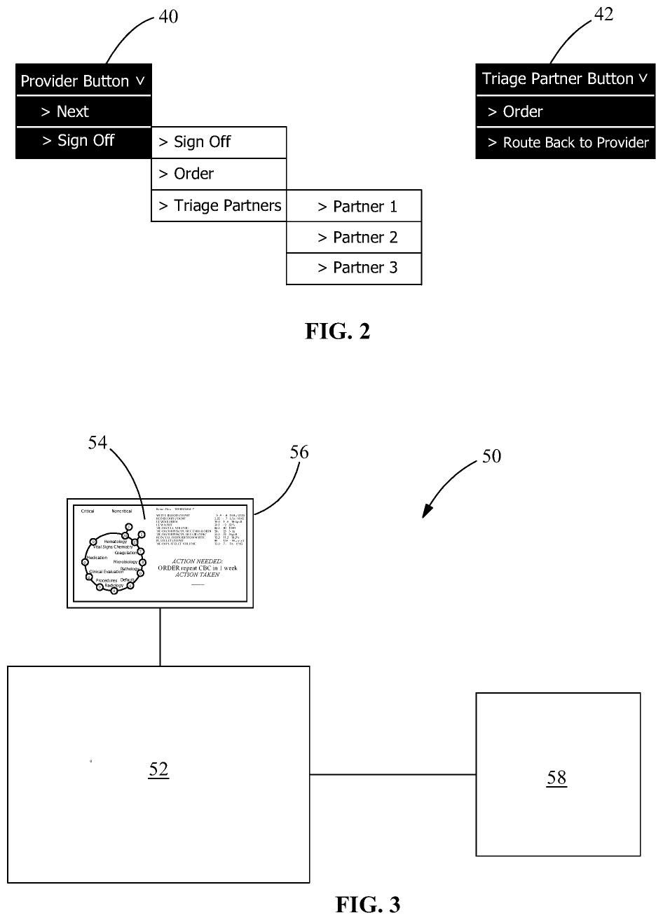

In exemplary embodiments, a triaged data display system facilitates the generation, tracking, and execution of a triage task. The computer prompts the designated user with a user menu 40 on the triaged data display, in the form of a dropdown menu in FIG. 2, to provide a designated user with the options described with respect to the process of FIG. 1. When the designated user selects the first option, “Next” in FIG. 2, the current report is moved to the bottom of the stack and the next report in the stack is displayed. When the designated user selects the second option, “Sign Off” in FIG. 2, a further portion of the user menu 40 appears with three additional options, “Sign Off”, “Order”, and “Triage Partners” in FIG. 2. When the designated user selects the first additional option, “Sign Off” in FIG. 2, the report is considered by the system to be signed off by the designated user with no further action being needed. When the designated user selects the second additional option, “Order” in FIG. 2, the triaged data display system links to a window where the designated user may perform a triage task, such as ordering a repeat of the test indicated in FIG. 1.

When the designated user selects the third additional option, “Triage Partners” in FIG. 2, a further portion of the dropdown menu 80 appears with potential triage partners being listed for selection. Although the triage partners are listed as “Partner 1”, “Partner 2”, and “Partner 3”, any number of appropriate triage partners may be listed, and they may be listed by any designation, such as, for example, a username, their first name, their last name, their first and last names, their initials, or a nickname. When the designated user selects one of the triage partners, the critical report 14 and the alert message 18 associated with this critical report 14 then become part of the triage partner's triaged data display 18 to be acted on by the triage partner.

In some embodiments, the designated user selects the appropriate alert message 14 for the triage task from a set of predetermined alert messages 14, such as, for example, in a drop-down menu. In other embodiments, the designated user is free to enter a custom message as the alert message 14 for the triage task.

The designated user may generate the alert message 14 within the user menu 40 of FIG. 2 or in a separate process within the triaged data display system. For example, when the designated user selects the second option, “Sign Off” in FIG. 2, the user menu 40 may provide the option of generating a triage task or generating an alert message 14. Alternatively, when the designated user selects the second additional option (“Order” in FIG. 2), the triaged data display system may prompt the designated user to input the alert text for the alert message 14 prior to directing the designated user to take a triage action. Similarly, when the designated user selects the third additional option (“Triage Partners” in FIG. 2), the triaged data display system may prompt the designated user to input the alert text for the alert message 14 either before or after permitting the designated user to select a triage partner to whom the triage task is to be assigned. The designated user who has generated the triage task is able to easily follow and monitor the status of the triage task in real time on the triaged data display 18 in an ongoing basis through the triaged data display system and process.

Still referring to FIG. 2, a partner menu 42, in the form of a dropdown menu in FIG. 2, provides the triage partner with options with respect to a triage task assigned to the triage partner. When the triage partner selects a first option, “Order” in FIG. 2, the triaged data display system links to a window where the designated user may perform a triage task, such as ordering a repeat of the test indicated in FIG. 1. When the triage partner selects a second option of the partner menu 42, “Route Back to Provider” in FIG. 2, the triaged data display system assigns the triage task back to the original designated user. A third option (not shown) may be provided to allow the triage partner to skip the current report and its associated triage task and move the report to the bottom of the stack.

Although the triage process 10 with respect to FIG. 1 and FIG. 2 is described for a critical report 14, a triaged data display system 50 as shown in FIG. 3 may similarly permit a designated user to initiate a triage task associated with a non-critical report.

The triaged data display system 50 may also include one or more reminder processes to promote completion of a triage task. These reminder processes may be automated by the triaged data display system 50 or may be based on user input. For example, when a triage task is initially established, the triaged data display system 50 may additionally send an electronic notification, such as, for example, a text message, a pager message, an electronic mail (e-mail) message, or a combination thereof, to an authorized device associated with the designated user and/or the triage partner. If the triage task remains incomplete after a predetermined period of time, the triaged data display system 50 may send an electronic reminder, such as, for example, a text message, a pager message, an e-mail message, or a combination thereof, to the designated user and/or the triage partner. The triaged data display system 50 may also send an electronic notification or an electronic reminder to the designated user or the triage partner based on an input from the designated user or the triage partner to the triaged data display system 50.

The triaged data display system is preferably computer-enabled and computer-implemented. Referring to FIG. 3, when computer-enabled, a triaged data display system 50 includes a local computer 52 enabling a triaged data display 54 to a local display monitor 56. In some embodiments, the local display monitor 56 is a user display monitor. The local computer 52 receives inputs from a designated user or a triage partner and executes a triage process. The local computer 52 may be on a network and in electronic or wireless communication with one or more server computers 58, where a database of the triaged data may be maintained, or with other local computers (not shown). In exemplary embodiments, the triaged data display system 50 includes a triaged data display program that implements a triage process 10.

A local computer 52, as used herein, may refer to a laptop computer, a desktop computer, an electronic tablet, a smart phone, or any other electronic device capable of processing data, receiving and processing designated user inputs, and directing the display of an image on an electronic display monitor 56.

In exemplary embodiments, a triaged data display system 50 provides a triaged data display 54 representing all data reports while highlighting the ones with critical results on a single screen. The triaged data display system 50 then enables triage of a critical report 14 by a designated user to a triage partner.

A critical report 14 remains on the triaged data display 54 of the designated user and is identified as triaged but not resolved, as discussed above. Only when the underlying problem noted in the critical report 14 is resolved, then the critical designation is removed. Additionally, the triaged data display 54 is preferably always displayed on a single screen.

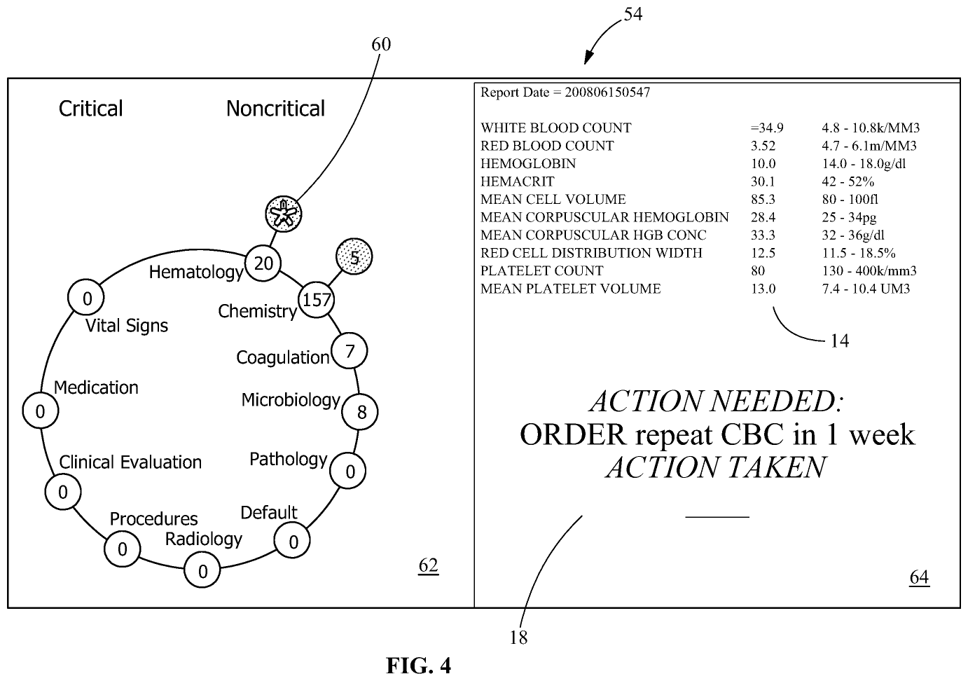

FIG. 4 shows a triaged data display 54 of 200 unacknowledged reports, limited to medical laboratory results, for this example. The triaged data display 54 is preferably shown on a display monitor 56 of a computer 52. The critical reports 14 are unacknowledged in that the designated user, in this case a health care professional, has not acknowledged to the triaged data display system 50 that those critical reports 14 have been reviewed.

In some embodiments, the reports are displayed as shown and described in U.S. Pat. No. 9,286,443, which is hereby incorporated by reference herein. Referring to the left portion 62 of the triaged data display 54 of FIG. 4, the reports are first divided into categories arrayed at fixed locations around a circle, such as, for example, the eleven categories shown in FIG. 4, with the numbers in the circles indicating the number of reports in that stack. The eleven categories are hematology, chemistry, coagulation, microbiology, pathology, default, radiology, procedures, clinical evaluation, medication, and vital signs, but any number or set of categories may be used. Within each category, the reports meeting a critical criterion are further partitioned in FIG. 4 as critical reports 14 enumerated in a small circle lying outside the larger circle and connected by a line to a small circle on the circumference of the larger circle. The partitioned corresponding non-critical reports in FIG. 4 are enumerated on the small circles on the circumference of the larger circle. Two prior lab repeats of the same test type may be included in the triaged data display 54 when merged with most EHR systems.

In exemplary embodiments, the placement of the cursor 60 over the small circle with the number “3” (critical “hematology” reports) displays the first report (“top of the stack”) on the right portion 64 of the triaged data display 54. The shown report is a critical report 14 in that at least one of the test results falls outside of a predefined normal range. The triaged data display 54 also provides the alert message 18 that was entered by the designated user of an action to be taken as a triage task as a result of the criticality of the critical report 14 and the designated user's review. That critical report 14 remains at the top of the critical stack or queue until a click signs off the report, or the report is triaged to a triage partner for “Action Needed”, as discussed above with respect to FIG. 1 and FIG. 2. After signoff of the top report, the next report in that data category queue automatically appears on the right portion 64 of the triaged data display 54, and the number in the small circle reduces by 1. Alternatively, the designated user may skip the top report on the stack, sending it to the bottom of the stack and causing the next report on the stack to be displayed, in which case the number in the small circle remains the same.

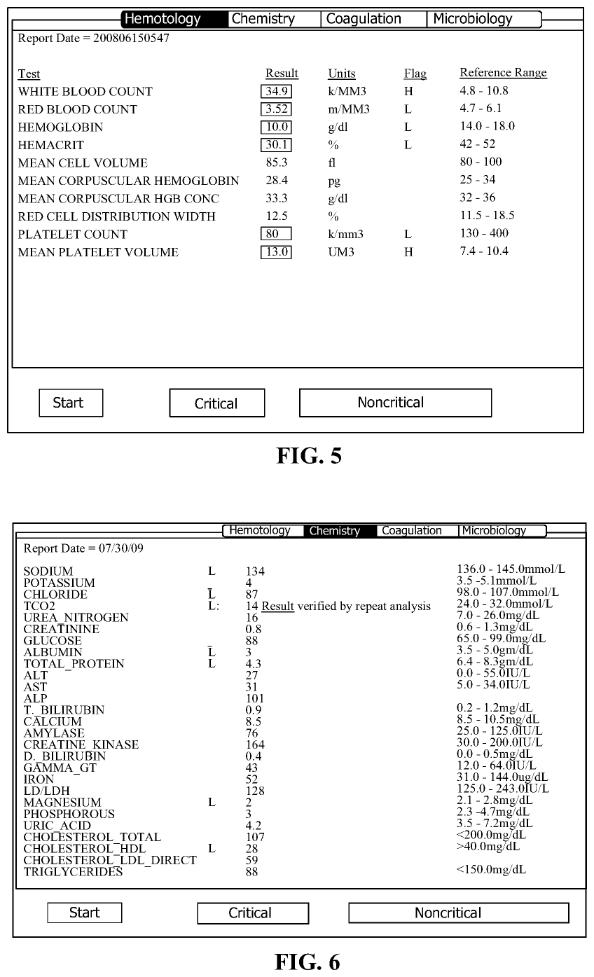

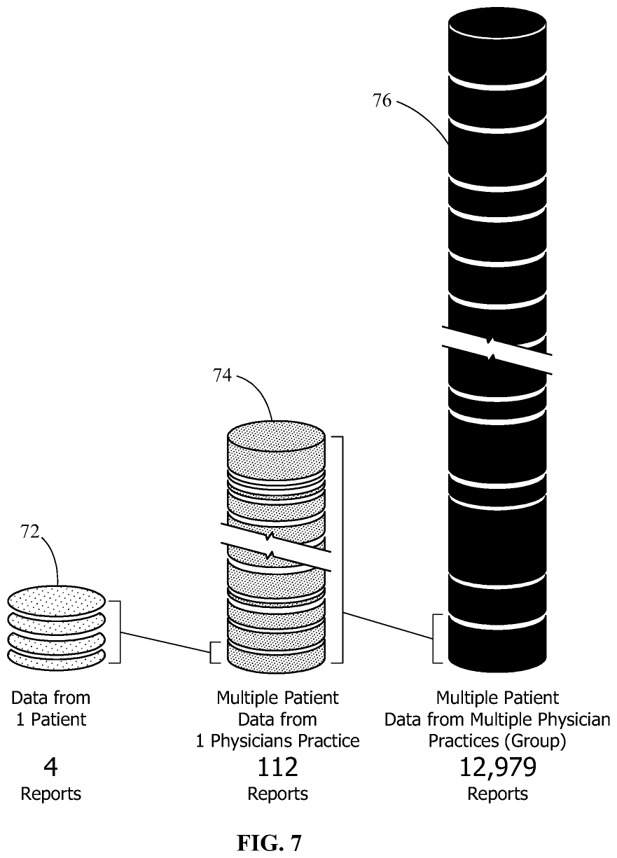

FIG. 5 shows a Meditech hematology result report (Medical Information Technology, Incorporated, Westwood, Mass.), shown here schematically with boxes around critical and abnormal but non-critical results, but critical results are typically flagged in a first color (such as, for example, red) and abnormal but non-critical results are typically flagged in a second color (such as, for example, yellow). FIG. 6 shows an Orchard® chemistry result report (Orchard Software Corporation, Carmel, Ind.), shown here without flagging of critical values, but critical values are typically flagged in a first color (such as, for example, red). Both of these display formats were tested in a clinical trial and found to be inferior to a display similar to the one shown in FIG. 4. The results of those tests were presented in “R.A.P.I.D. (Root Aggregated Prioritized Information Display): A single screen display for efficient digital triaging of medical reports”, Ford et al., Journal of Biomedical Informatics, Vol. 61, pp. 214-223 (2016), which is hereby incorporated by reference herein.

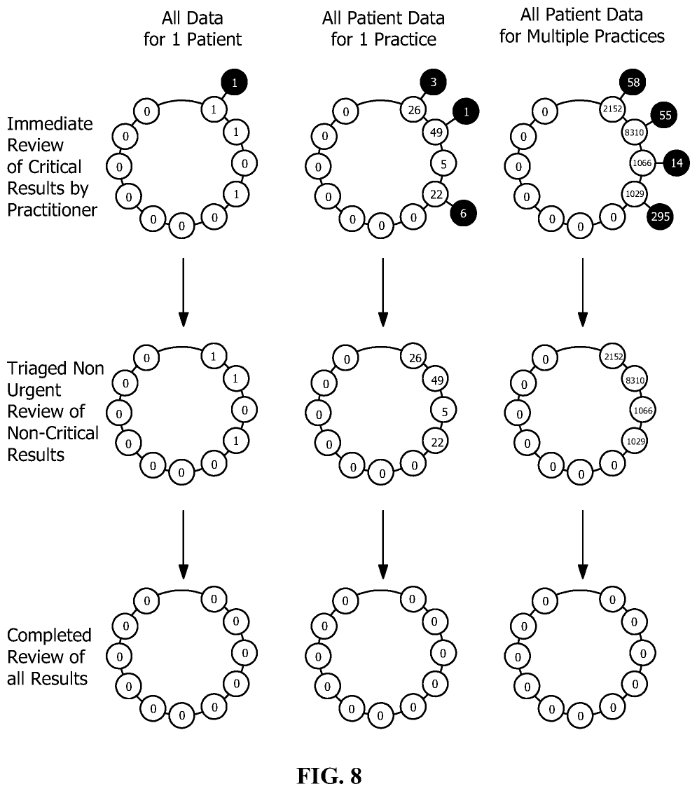

In exemplary embodiments, a triaged data display 54 includes a constant display format for all of the data of a single patient, multiple patients, or even multiple practices. These three different scenarios are illustrated schematically as a single patient stack 72, a multi-patient single practice stack 74, and a multi-patient, multi-practice stack 76 in FIG. 7. The constant triaged data display 54 view at the top of the stack (FIG. 8, top row) enumerates and represents all of the reports within the underlying queue. In this example, the category assignments represented by each circle are constant and labeled in FIG. 4. The left column represents part of the display for four reports of a single individual as a single patient stack 72. The center column represents part of the display of 112 reports for a single practice as a multi-patient single practice stack 74. The right column represents part of the display of 12,979 reports from multiple practices as a multi-patient, multi-practice stack 76. The increasing height and grey scale of the cylinders of FIG. 7 schematically reflects the increasing data density going from left to right. The reports are understood to be in a virtual “stack”.

FIG. 8 shows how a triaged data display 54 improves workflow in medical data management. Critical reports 14 are acknowledged immediately by the responsible practitioner or provider (“designated user”), typically by generating a triage task to be handled by the designated user or sent to another member of the health-care team (“triage partners”). This modifies the stacks 72, 74, 76 from the top row version to the middle row version of FIG. 8. Non-critical reports are acknowledged routinely by the provider (“designated user”) and may also be triaged by the designated user or assigned to other members of the health-care team (“triage partners”). This further modifies the stacks 72, 74, 76 from the middle row version to the bottom row version of FIG. 8, where the designated user has now dealt with all outstanding reports.

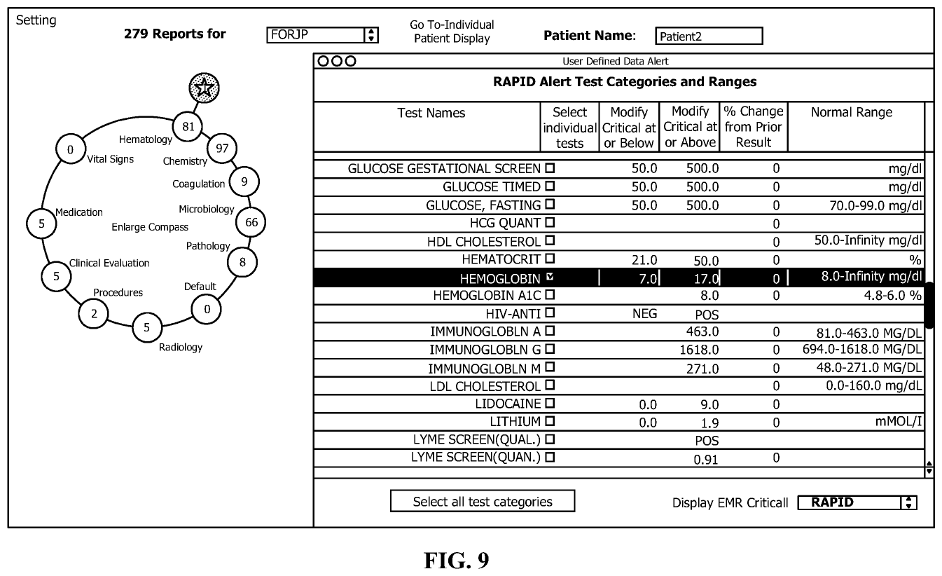

Referring to FIG. 9, the triaged data display system 50 may be adaptable to permit a designated user to adjust the criteria that define critical information for the triaged data display 54. This may include a fixed magnitude change in the values that define a normal test result, which may be based on a prior result, as shown for the hemoglobin level 80 in FIG. 9. This may cause the triaged data display system 50 to recategorize a report as critical or non-critical.

Although the triage process 10 and the triaged data display system 50 have been described with the designated user being a health care professional, the designated user may instead be patient. In some embodiments where the designated user is a patient, all laboratory and clinical medical data for that patient and no other patients is displayed by the triaged data display system 50. In such embodiments, the primary health care provider for the patient may serve as the triage partner.

Embodiments where the patient is the designated user may have a number of advantages over embodiments where a health care professional is the designated user. First, all of the laboratory and clinical data collected from any source from the patient is more available to primary provider through the patient. Often, if a patient goes to a health care facility not connected to the primary provider, the data is unavailable to the primary provider or delivered with unsafe delays. Second, the patient may be reassured and calmed that the primary provider is notified of all issues that are determined to be important.

Third, the primary provider can set the critical ranges to suit the practice's pattern. Often a “critical” result is a result for a specific patient that is a significant change from a recent prior result for the patient, such as, for example, a 20% decrease in hemoglobin. Some patients with chronic illnesses have consistently very abnormal but stable laboratory values, and such results are not “critical” for that patient. Typically, clinical laboratories can only report as “critical” based on the general population, when laboratory results are above or below set general values.

Fourth, most, substantially all, or all HIPAA issues are resolved with the patient as the designated user, because the patient is releasing the health information and thus giving consent to its use.

Fifth, the common display format of laboratory/clinical result as delivered to patients makes it easy to develop an interface to the triaged data display 54, in contrast to the data from the various incompatible proprietary electronic medical record systems provided to hospitals and provider practices.

Sixth, the patient as the designated user may also enter clinical data, such as, for example, body temperature, nausea symptoms, vomiting incidents, or diarrhea incidents, that the primary provider as triage partner may then evaluate and establish as critical on the basis of severity thresholds.

Finally, with the patient as the designated user, the triaged data display system 50 may include a time feature, where the triaged data display system 50 triggers a critical event on the triaged data display 54 of the patient as designated user and/or the primary provider as triage partner when the patient, who is on an active treatment, has not been in contact with the primary provider for a predetermined period of time.

In exemplary embodiments, the triaged data display system 50 is adapted to interface with a pre-existing health record system, such as, for example, the Epic health record system (Epic Systems Corporation, Verona, Wis.) currently being used at Guthrie Clinic (Sayre, Pa.).

Although the examples described herein have been in the field of patient medical data, a triaged data display system 50 may be used in any field where reports or results may precipitate or require further actions of varying urgencies. What defines a critical result in medicine is often a very abnormal numeric value.

In other fields, it might be module completion of a complex project. For example, a major corporation was recently implementing a total software change in Human Relations (HR). Unfortunately, the project was delayed because implementation of the payroll module lagged behind implementation of the other modules. In that scenario, a triaged data display system 50 may display the set of modules around the circle with relevant categories. The project manager (“designated user”) may then monitor task completion of the individual modules. As the component tasks within a module are submitted as complete, the project manager may confirm the module as finished, which decreases the number of remaining critical tasks in that module stack by one. If completion of one module is lagging, the project manager may reassign or redistribute triage tasks to different team members (“triage partners”) and monitor the completion of those triaged tasks. In another embodiment, the system monitors the status of a set of machines in a complex manufacturing facility or multiple facilities. In another example, a triaged data display system 50 may be used in the legal field for tracking and monitoring progress toward completion of various triaged tasks, such as, for example, court filings with deadlines.

The above are only some examples of a myriad of possible embodiments. A triaged data display system 50 may be useful in virtually any setting where a high standard of performance is required of a system with complex, numerous components. In exemplary embodiments, a triaged data display system 50 promotes higher work efficiency and improved team communication within a group of users.

In exemplary embodiments, the local computer 52, the server computer 58, and any other electronic devices in electronic communication as part of the triaged data display system 50 include respective sets of internal components and external components. Each of the sets of internal components may include one or more operating systems, one or more computer-readable tangible storage devices, one or more processors, one or more computer-readable random access memories (RAMs), and one or more computer-readable read-only memories (ROMs) on one or more buses. The operating system and a triaged data display program are stored on one or more of the computer-readable tangible storage devices for execution by one or more of the processors via one or more of the RAMs. Each of the computer-readable tangible storage devices may be a magnetic disk storage device of an internal hard drive or a semiconductor storage device such as ROM, erasable programmable ROM (EPROM), flash memory, or any other computer-readable tangible storage device that can store a computer program and digital information.

Each set of internal components also includes a read/write (R/W) drive or interface to read from and write to one or more portable computer-readable tangible storage devices, such as a compact disc read-only memory (CD-ROM), digital versatile disc (DVD), memory stick, magnetic tape, magnetic disk, optical disk, or semiconductor storage device. The triaged data display program may be stored on one or more of the portable computer-readable tangible storage devices, read via R/W drive, or interface and loaded into a hard drive.

Each set of internal components may also include a network adapter or interface such as a transmission control protocol/internet protocol (TCP/IP) adapter card. The triaged data display program may be downloaded to the local computer 52 and/or server computer 58 from an external computer via a network (for example, the Internet, a local area network (LAN) or other, wide area network (WAN)) and network adapter or interface. From the network adapter or interface, the triaged data display program may be loaded into the hard drive. The network may include copper wires, optical fibers, wireless transmission, routers, firewalls, switches, gateway computers, and/or edge servers.

Each of the sets of external components may include a display monitor 56, a keyboard, and/or a computer mouse. Each of the sets of internal components may also include device drivers to interface to the display monitor, the keyboard, and the computer mouse. The device drivers, R/W drive, or interface and network adapter or interface include hardware and software stored in a storage device and/or ROM.

The triaged data display program may be written in any of various programming languages including low-level, high-level, object-oriented, or non-object-oriented languages. Alternatively, the functions of the triaged data display program may be implemented in whole or in part by computer circuits and other hardware (not shown).

A triaged data display program product may include a computer-readable storage medium (or media) having computer-readable program instructions thereon for causing a processor to carry out aspects of the triage process described herein.

The computer-readable storage medium may be a tangible device that retains and stores instructions for use by an instruction execution device. The computer-readable storage medium may be, for example, an electronic storage device, a magnetic storage device, an optical storage device, an electromagnetic storage device, a semiconductor storage device, or any suitable combination of the foregoing. More specific examples of appropriate computer-readable storage media include a portable computer diskette, a hard disk, a RAM, a ROM, an EPROM, a static random access memory (SRAM), a portable CD-ROM, a DVD, a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media, or electrical signals transmitted through a wire.

Computer-readable program instructions may be downloaded to respective computing/processing devices from a computer-readable storage medium or to an external computer or external storage device via a network, for example, the Internet, a LAN, a WAN, and/or a wireless network. The network may include copper transmission cables, optical transmission fibers, wireless transmission, routers, firewalls, switches, gateway computers, and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the triaged data di splay system may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object-oriented programming language, such as Smalltalk or C++, and procedural programming languages, such as the “C” programming language or similar programming languages. The computer-readable program instructions may execute entirely on the local computer 52, partly on the local computer 52, as a stand-alone software package, partly on the local computer 52 and partly on a server computer 58, or entirely on the server computer 58. In the latter scenario, the server computer 58 may be connected to the local computer 52 through any type of network, including a LAN or a WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA), may execute the computer-readable program instructions by utilizing state information of the computer-readable program instructions to personalize the electronic circuitry, in order to perform aspects of the triage process 10.

It will be understood that each aspect of the triage process 10 may be implemented by computer-readable program instructions. These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing steps of the triage process 10. These computer-readable program instructions may also be stored in a computer-readable storage medium that may direct a computer, a programmable data processing apparatus, and/or other devices to function in a particular manner, such that the computer-readable storage medium having instructions stored therein includes an article of manufacture including instructions that implement aspects of the triage process.

The computer-readable program instructions may also be loaded onto a computer, other programmable data processing apparatus, or other device to cause a series of operational steps to be performed on the computer, other programmable apparatus, or other device to produce a computer-implemented process, such that the instructions that execute on the computer, other programmable apparatus, or other device implement the specified functions/acts of the triage process 10.

Although the triage process and triaged data display system have been described with respect to medical records and for use in the medical field, the principles of the process and system may be implemented in other ways as well. In some embodiments, a triaged data display system is implemented in a more modular way, such that the user interface (UI) may be used by different companies, such as, for example, a data visualization plugin for data analysis tools or spreadsheets, such as, for example, Microsoft Excel and Tableau. In some embodiments, the triage function is implemented separately, using a modular triaged data display UI. As such, the modular triaged data display UI may be applied to any data field for many different applications, such as, for example, genomic data display, whereas the full triaged data display system including triage functions may be used, for example, by hospitals, warehouses, human resource departments, or the military.

All above-mentioned references are hereby incorporated by reference herein.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A triage process comprising:
a computer directing a user triaged data display to appear on a user display monitor of a designated user;
the computer prompting the designated user through the user triaged data display to initiate a triage task associated with a first report appearing on the user triaged data display;
the computer prompting the designated user through the user triaged data display to assign the triage task to a triage partner; and
the computer directing an alert message associated with the triage task to appear as part of a partner triaged data display on a partner display monitor of the triage partner based on inputs from the designated user.

2. The triage process of claim 1 further comprising the computer prompting the designated user to select the triage partner.

3. The triage process of claim 1 further comprising the computer prompting the designated user to provide the alert message.

4. The triage process of claim 1 further comprising the computer categorizing a plurality of reports comprising the first report into a plurality of categories and partitioning the plurality of reports into non-critical reports and critical reports.

5. The triage partner of claim 4 further comprising the computer directing the user triaged data display to display the plurality of reports as a plurality of stacks with a non-critical stack of non-critical reports for each category appearing at a predetermined location around a circle and a critical stack of critical reports for each category lying outside the circle.

6. The triage process of claim 1 further comprising the computer directing a triage icon to appear on the user triaged data display, the triage icon providing a visual indication of a current status of the triage task based on inputs from the triage partner.

7. The triage process of claim 1 further comprising the computer directing a reminder to the user triaged data display or the partner triaged data display when the triage task remains incomplete for a predetermined period of time.

8. The triage process of claim 1, wherein the first report is a critical report.

9. The triage process of claim 1, wherein the designated user is a health care professional and the triage partner is a health care specialist.

10. The triage process of claim 1, wherein the designated user or the triage partner is a patient.

11. The triage process of claim 10 further comprising prompting the patient for permission to share the first report with a health care provider and sharing the first report with the health care provider upon receiving permission from the patient.

12. The triage process of claim 1, wherein the user triaged data display provides an interface for the designated user to access all data in a database to which the designated user has authorized access, the database comprising a plurality of reports comprising the first report.

13. A triaged data display system comprising:
a user display monitor; and
a user computer operatively connected to the user display monitor, the user computer configured to:
direct a user triaged data display to appear on the local display monitor or a user display monitor of a designated user;
prompt the designated user through the user triaged data display to initiate a triage task associated with a first report appearing on the user triaged data display;
prompt the designated user through the user triaged data display to assign the triage task to a triage partner; and
direct an alert message associated with the triage task to a partner computer configured to direct the alert message and the triage task to appear as part of a partner triaged data display on a partner display monitor of the triage partner based on inputs from the designated user.

14. The triaged data display system of claim 13 further comprising the partner computer operatively connected to the partner display monitor.

15. The triaged data display system of claim 13, wherein the user computer is further configured to prompt the designated user to select the triage partner.

16. The triaged data display system of claim 13, wherein the user computer is further configured to prompt the designated user to provide the alert message.

17. The triaged data display system of claim 13, wherein the user computer is further configured to categorize a plurality of reports comprising the first report into a plurality of categories and partitioning the plurality of reports into non-critical reports and critical reports.

18. The triaged data display system of claim 17, wherein the user computer is further configured to direct the user triaged data display to display the plurality of reports as a plurality of stacks comprising a non-critical stack of non-critical reports for each category appearing at a predetermined location around a circle and a critical stack of critical reports for each category lying outside the circle.

19. The triaged data display system of claim 17, wherein the plurality of reports share a common display format.

20. The triaged data display system of claim 13, wherein the user computer is further configured to direct a triage icon to appear on the user triaged data display, the triage icon providing a visual indication of a current status of the triage task based on inputs from the triage partner.

21. The triaged data display system of claim 13, wherein the user computer is further configured to direct a reminder to the user triaged data display or the partner triaged data display when the triage task remains incomplete for a predetermined period of time.
